(19) United States
(12) Patent Application Publication
(10) Pub. No.: US 2006/0166982 A1
(43) Pub. Date: Jul. 27, 2006

(54) Isotopically labelly indlinone derivatives and process for their preparation

(76) Inventors: Danilo Giribone, Carozzi (IT); Alberto Pignatti, Milan (IT); Erminia Fontana, Garibaldi (IT)

Correspondence Address: SCULLY SCOTT MURPHY & PRESSER, PC, 400 GARDEN CITY PLAZA, SUITE 300, GARDEN CITY, NY, 11530, US

(21) Appl. No.: 10/523,276

(22) PCT Filed: Jul. 28, 2003
(86) PCT No.: PCT/EP03/05040

§ 371 Date: Sep. 19, 2005

(30) Foreign Application Priority Data
Aug. 1, 2002 (EP) 02078164.7

(51) Int. Cl. A61K 31/5377; A61K 31/454; A61K 31/404; C07D 413/14; C07D 403/14

(52) U.S. Cl. 514/232.5; 514/323; 514/414; 546/201; 548/465; 544/153

(57) ABSTRACT

Compounds which are isotopically labeled carbonium 14 [¹⁴C] indolinone derivatives and process for their preparation are disclosed; these compounds are useful for absorption, distribution, metabolism and excretion (ADME) studies.

The present invention relates to indolinone derivatives and, more particularly, it relates to indolinone compounds isotopically labelled with carbonium 14 [¹⁴C], and to a process for their preparation.

Several indolinone derivatives are known in the art as therapeutic agents.

Particularly relevant, among them, are certain (1,2-dihydro-2-oxo-3H-indol-3-ylidene)methyl-1H-pyrrole derivatives, hereinafter shortly referred to as indolylidene-methyl-pyrroles, disclosed by Sugen Inc. in a variety of patents and patent applications, among which are U.S. Pat. No. 5,880,141, U.S. Pat. No. 5,792,783, WO 96/40116, WO 99/48868, WO 99/61422, WO 01/37820, WO 02/66463 and WO 03/35009, herewith incorporated by reference.

By modulating tyrosine kinase signal transduction, the said compounds are useful in therapy for regulating, modulating and/or inhibiting abnormal cell proliferation.

Because of their use in therapy, for instance in the treatment of cancer, the possibility of their preparation as isotopically labelled carbonium 14 [¹⁴C] compounds is of utmost importance for absorption, distribution, metabolism and excretion (ADME) studies.

From the above, we have now found a new class of indolylidene-methyl-pyrroles being isotopically labelled with [¹⁴C] at the methylidene moiety.

It is therefore a first object of the present invention a compound of general formula (I) below: 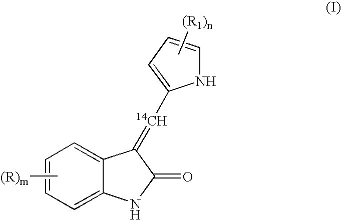
wherein
each R group is, at one or more of the positions 4, 5, 6 and 7 of the indolinone ring and independently from each other, a straight or branched C₁-C₄ alkyl or alkoxy group or a halogen atom;
each R₁ group is, the same or different and at one or more of the positions of the pyrrole ring, a C₁-C₄ alkyl or a group of general formula —(CH₂)ₚCO₂R′, —(CH₂)ₚ—CONR′R″ or —CONH—(CH₂)ₚ—CONR′R″ wherein p is 0, 1, 2 or 3, the alkylene —(CH₂)ₚ— chain is optionally substituted by hydroxy, and R′ and R″ are selected, each independently, from hydrogen or straight or branched C₁-C₄ alkyl optionally substituted by hydroxy or, taken together with the nitrogen atom to which they are attached, R′ and R″ may form a pyrrolidino, piperidino or morpholino group;
m is 0 or an integer from 1 to 4;
n is 0 or an integer from 1 to 3;
   or pharmaceutically acceptable salts thereof.

As clearly reported in formula (I), labelling with ¹⁴C occurs at the methylidene moiety bridging the indolinone with the pyrrole ring.

The compounds of formula (I) may have asymmetric carbon atoms and may therefore exist either as racemic mixtures or as individual optical isomers. In addition, the double bond in general formula (I) between the carbon atom in position 3 of the indolinone ring and the labelled [¹⁴C] atom, may be such to give rise to any one of the cis (Z) or trans (E) isomers.

From the foregoing and unless otherwise provided, all of the optical or geometrical isomers as well as the mixtures thereof, have to be intended as comprised within the scope of the present invention.

Unless otherwise provided, in the present description, with the terms straight or branched C₁-C₄ alkyl or alkoxy group we intend, for instance, methyl, ethyl, n-propyl, isopropyl, n-butyl, isobutyl, sec-butyl, tert-butyl, methoxy, ethoxy, n-propoxy, isopropoxy, n-butoxy, isobutoxy, sec-butoxy and tert-butoxy.

With the term halogen atom we intend a fluorine, chlorine, bromine or iodine atom.

Pharmaceutically acceptable salts of the compounds of formula (I) are the acid addition salts with inorganic or organic acids, e.g. nitric, hydrochloric, hydrobromic, sulphuric, perchloric, phosphoric, acetic, trifluoroacetic, propionic, glycolic, lactic, oxalic, malonic, malic, maleic, tartaric, citric, benzoic, cinnamic, mandelic, methanesulfonic, isethionic and salicylic acid, as well as the salts with inorganic or organic bases, e.g. alkali or alkaline-earth metals, especially sodium, potassium, calcium or magnesium hydroxides, carbonates or bicarbonates, acyclic or cyclic amines, preferably methylamine, ethylamine, diethylamine, triethylamine or piperidine.

As formerly indicated, the indolinone derivatives of the invention may be optionally substituted by R groups in one or more of the positions 4, 5, 6 and 7, according to the numbering system below:

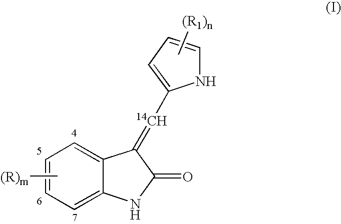

Likewise, the indolinone derivatives of the invention may be also optionally substituted in one or more of the free positions of the pyrrole ring by the above R₁ groups.

Preferably, the compounds of the invention may be represented by the above general formula (I) wherein the pyrrole ring is substituted by one or more groups such as, for instance, methyl, carboxy, ethoxycarbonyl, carboxyethyl, N,N-diethyl-aminocarbonyl, N-[(2-diethylamino)ethyl]carboxamide, N-[2-hydroxy-3-morpholin-4-ylpropyl]carboxamide, and the like.

Even more preferably, the compounds of the invention are selected from 3-[(3,5-dimethyl-1H-pyrrol-2-yl)[¹⁴C]methylene-1,3-dihydro-2H-indol-2-one (hereinafter shortly referred to as [¹⁴C]SU-5416); 5-[(1,2-dihydro-2-oxo-3H-indol-3-ylidene)[¹⁴C]methyl]-2,4-dimethyl-1H-pyrrole-3-propionic acid (hereinafter shortly referred to as [¹⁴C]SU-6668); N-[(2-diethylamino)ethyl]-5-[(5-fluoro-1,2-dihydro-2-oxo-3H-indol-3-ylidene)[¹⁴C]methyl]-2,4-dimethyl-1H-pyrrole-3-carboxamide (hereinafter shortly referred to as [¹⁴C]SU-11248); 3-{5-methyl-2-[(Z)-(2-oxo-1,2-dihydro-3H-indol-3-ylidene) [¹⁴C]methyl]-1H-pyrrol-3-yl)}propanoic acid (hereinafter shortly referred to as [¹⁴C]SU-10944); and 5-[(Z)-(5-fluoro-2-oxo-1,2-dihydro-3H-indol-3-ylidene) [¹⁴C]methyl]-N-[(2S)-2-hydroxy-3-morpholin-4-ylpropyl]-2,4-dimethyl-1H-pyrrole-3-carboxamide (hereinafter shortly referred to as [¹⁴C]SU-14813), of formula:

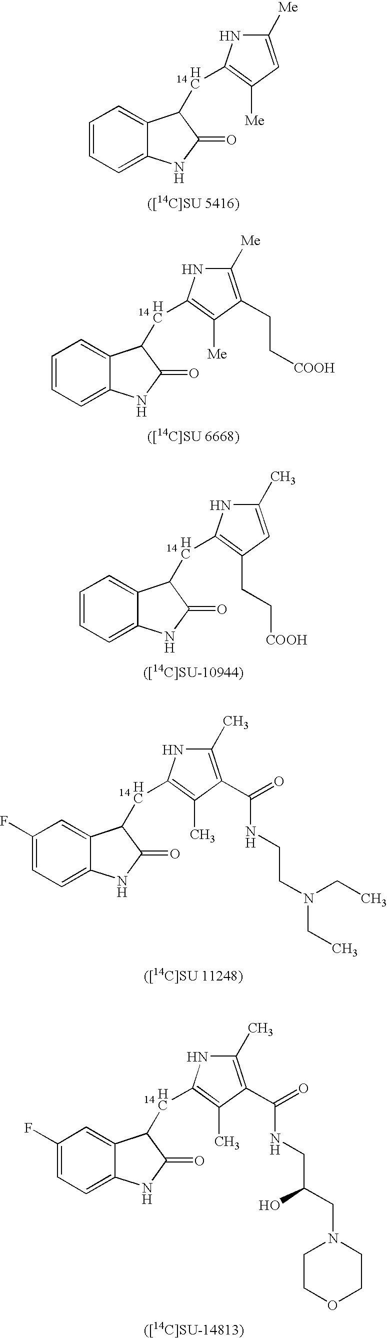

As formerly indicated, it is another object of the invention a process for preparing the compounds of formula (I) and the pharmaceutically acceptable salts thereof, which process comprises:
a) reacting dimethyl-[¹⁴C]formamide with a suitable pyrrole derivative of formula (II), in the presence of diphosphoryl-chloride 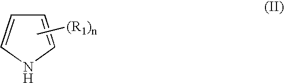
wherein R₁ and n are as above defined, so as to obtain a compound of formula (III) 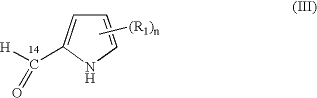
and optionally converting a compound of formula (III) into another compound of formula (III);
b) reacting under basic conditions the compound of formula (III) with an oxindole derivative of formula 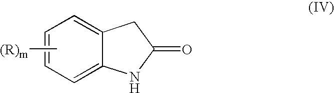
wherein R and m are as above defined, so as to obtain a compound of formula (I) and, optionally converting it into another compound of formula (I) and/or into a pharmaceutically acceptable salt thereof.

The above process is particularly advantageous as it enables the selective preparation of a variety of compounds of formula (I) isotopically labelled with [¹⁴C], being optionally substituted with several R and R₁ groups on both the indolinone and/or pyrrole moieties.

In addition, it enables the preparation of the desired derivatives in high yields and with a high degree of radiochemical purity.

According to step (a) of the process, dimethyl-[¹⁴C]formamide is reacted with a proper pyrrole derivative, either substituted or unsubstituted by R₁ groups, as formerly indicated. The reaction is carried out under inert atmosphere, e.g. nitrogen or argon, in the presence of diphosphoryl chloride, at a temperature ranging from about oIC to about room temperature and for a time of about 40 minutes.

As formerly indicated, the compounds of formula (III) thus prepared may be conveniently converted into others compounds of formula (III), for instance by transforming a given R′ group into another R′ group. As an example, a compound of formula (III) bearing an ester R₁ group, e.g. —(C₂)ₚCO₂R′ with R′ as alkyl, may be conveniently converted into the corresponding carboxylic acid derivative wherein R′ is hydrogen.

The above reaction may be either carried out subsequently to the preparation of the compound of formula (III) or, advantageously, in one pot without the need of isolating any intermediate derivative. Any of the above conversions may be carried out according to well known methods.

As an example, the conversion of an ester group into the corresponding carboxylic acid derivative may be easily accomplished through basic hydrolysis, for instance in the presence of potassium hydroxide under water/methanol refluxing conditions.

Likewise, any of the above derivatives of formula (III) bearing a R₁ group corresponding to —(C₂)ₚCO₃H may be also converted, whenever desired, into the corresponding carboxamido derivatives —(CH₂)ₚ—CONR′R″ or —CONH—(CH₂)ₚ—CONR′R″. Also the above reactions are performed according to conventional conditions for preparing carboxamides, for instance by reacting a suitable carboxylic acid derivative of formula (III) with the proper amino derivative, in the presence of benzotriazol-1-ylotris(dimethylamino)phosphonium hexafluorophosphate (BOP) and of a tertiary amine, e.g. triethylamine.

The reaction may occur in the presence of a suitable solvent, e.g. dimethylformamide, and at room temperature. According to step (b) of the process, any of the above compounds of formula (III) is reacted, under basic conditions, with a suitable indolinone derivative of formula (IV). This condensation reaction is carried out according to conventional methods, in the presence of catalytic amounts of a suitable base, e.g. pyrrolidine, and in a suitable solvent, e.g. ethanol, at refluxing conditions and for a suitable time, e.g. from about 30 to about 90 minutes.

By working as above reported in step (a) when converting a compound of formula (III) into another derivative of formula (III), also the compounds of formula (I) being obtained in step (b) may be conveniently converted into other derivatives of formula (I).

As an example, any given compound of formula (I) wherein R₁ is an ester group may be converted into the corresponding derivative of formula (I) wherein R₁ may represent a carboxy and/or carboxamido group, as formerly described.

Likewise, the optional salification of a compound of formula (I) or the conversion of its salt into the free compound, as well as the separation of a mixture of isomers into the single isomers, may be all carried out by conventional methods.

The starting dimethyl-[¹⁴C]formamide is a commercially available compound and any of the derivatives of formula (II) and (IV) is known or may be prepared according to well-known synthetic methods.

According to a preferred embodiment of the invention, the above process is addressed to the preparation of the aforementioned isotopically [¹⁴C] labelled indolinone derivatives SU 5416, SU 6668, SU 11248, SU-10944 and SU-14813.

In this respect, any of the intermediate derivatives of formula (IIIa) or (IIIb) below 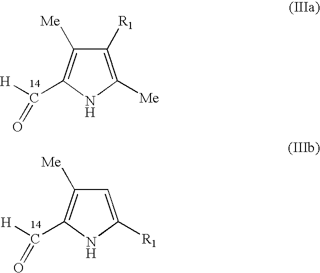
wherein R₁ is a hydrogen atom or a group —(CH₂)₂—CO₂H, —CO₂H, —CO₂CH₂CH₃, —CONH— (CH₂)₂—N(CH₂CH₃)₂ and 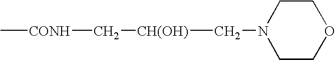
are novel and, hence, represent a further object of the invention.

The isotopically [¹⁴C] labelled indolinone derivatives of formula (I) may be used in ADME studies according to conventional methods, widely known in the art.

With the aim of better illustrate the present invention, without posing any limitation to it, the following examples are now given.

### EXAMPLE 1

### Preparation of 3,5-dimethyl-1H-pyrrole-2-[¹⁴C]carbaldehyde

Dimethyl[¹⁴C]formamide (about 740 MBq, 1.045 mmol) was cooled with an ice bath and very slowly added via a syringe with diphosphoryl chloride (DPC) (380 μl; 2.76 mmol). After stirring at about 0° C. under nitrogen atmosphere for 10 minutes, 2,4 dimethylpyrrole (130 μl; 1.275 mmol) was added to the solution over a period of 10 minutes and the mixture was stirred for 30 minutes at room temperature. At the end of reaction (checked by radio-HPLC on C-18 reverse phase column along with eluants as mixtures of water-acetonitrile-trifluoroacetic acid from 90:10:0.1 to 10:90:0.1 by volume, linear gradient over 15 minutes and 5 minutes of isocratic elution, detection wavelength=255 nm, radiometric detection=homogeneous with a 500 μl cell and scintillation cocktail to HPLC effluent ratio of 2:1 by volume) the mixture was cooled at −10° C. and a solution of methanol:water 1:5 v:v (3 ml) was introduced into the flask. After adjusting the pH to about 12 by addition of a 10% solution of potassium hydroxide, a white suspension was obtained which was filtered through a D4 sintered-glass filter and washed with water (4×3 ml). The solid 3,5-dimethyl-1H-pyrrole-2-[¹⁴C]carbaldehyde was obtained as a white solid (360 MBq), 95% radiochemically pure. The radiochemical purity of the title compound was assessed by radio-HPLC (on C-18 reverse phase column along with eluants as mixtures of water-acetonitrile-trifluoroacetic acid from 90:10:0.1 to 10:90:0.1 by volume, linear gradient over 15 minutes and 5 minutes of isocratic elution, detection wavelength=255 nm, radiometric detection=homogeneous with a 500 μl cell and scintillation cocktail to HPLC effluent ratio of 2:1 by volume), the retention time of title compound (Rt=9.11 minutes) was the same as the retention time of an authentic non-labelled sample. The radiochemical yield of this step was about 49%.

### EXAMPLE 2

### Preparation of 3-[(3,5-dimethyl-1H-pyrrol-2-yl)[¹⁴C]methylene]-1,3-dihydro-2H-indol-2-one ([¹⁴C]SU 5416)

3,5-dimethyl-1H-pyrrole-2-[¹⁴C] carbaldehyde (about 360 MBq; 0.48 mmol prepared as described, for instance in example 1, and oxindole (64.3 mg; 0.48 mmol) were dissolved with ethanol (3 ml). Pyrrolidine (70 μl; 1.71 mmol) was then added and the solution was stirred at reflows for 90 minutes in the dark. The obtained suspension was cooled at room temperature and filtered through a D4 sintered-glass filter giving a yellow-red solid that was washed with ethanol (4×3 ml). After drying, 3-[(3,5-dimethyl-1H-pyrrol-2-yl)[¹⁴C]methylene]-1,3-dihydro-2H-indol-2-one ([¹⁴C]SU 5416) was obtained (about 194 MBq; 0.261 mmol) 99% radiochemically pure. The radiochemical purity was assessed by radio-HPLC (on C-18 reverse phase column along with eluants as mixtures of water-acetonitrile-trifluoroacetic acid from 90:10:0.1 to 10:90:0.1 by volume, linear gradient over 15 minutes and 5 minutes of isocratic elution, detection wavelength=255 nm, radiometric detection=homogeneous with a 500 [i cell and scintillation cocktail to HPLC effluent ratio of 2:1 by volume), the retention time of title compound (Rt=15.4 minutes) was the same as the retention time of an authentic non-labelled sample. The mass spectrum of the title compound was recorded using the electrospray ionization technique (ESI) with positive ion detection. The ESI mass spectrum showed the protonated molecular ions at m/z 241 of 3-[(3,5-dimethyl-1-pyrrol-2-yl)[¹⁴C]methylene]-1,3-dihydro-2H-indol-2-one and also the protonated molecular ions at m/z 239 of 3-[(3,5-dimethyl-1H-pyrrol-2-yl)methylene]-1,3-dihydro-2H-indol-2-one. The radiochemical yield of this step was about 54%.

### EXAMPLE 3

### Preparation of 3-(3,5-dimethyl-2-[¹⁴C]formyl-1H-pyrrol-4-yl)-propionic acid

Dimethyl[¹⁴C]formamide (about 740 MBq, 1.045 mmol) was cooled with an ice bath and very slowly added via a syringe with DPC (900 μl). After 10 minutes of stirring, the above cooled (ice bath) solution was added with 3-(2,4-dimethyl-1H-pyrrol-3-yl)propanoic acid (213 mg, 1.27 mmol) over 15 minutes under nitrogen, then allowed to warm to room temperature and the mixture was stirred for 30 minutes at room temperature. At the end of reaction checked by radio-HPLC (on C-18 reverse phase column along with eluants as mixtures of water-acetonitrile-trifluoroacetic acid from 90:10:0.1 to 10:90:0.1 by volume, linear gradient over 15 minutes and 5 minutes of isocratic elution, detection wavelength=255 nm, radiometric detection=homogeneous with a 500 μL cell and scintillation cocktail to HPLC effluent ratio of 2:1 by volume), the mixture was cooled at −10° C., a solution of methanol:water 1:5 v:v (3 ml) was added. After adjusting the pH to about 12 by addition of a 45% solution of potassium hydroxide, the solution was stirred at 0° C. for 30 minutes. The suspension was filtered through a D4 sintered-glass filter obtaining a yellow clear solution, which was added with 10 N solution of hydrochloric acid up to pH 3.5. The mixture was stirred at 0° C. for 30 minutes. The resulting brown suspension was filtered through a D4 sintered-glass filter, the intermediate 3-(3,5-dimethyl-2-[¹⁴C] formyl-1H-pyrrol-4-yl)-propionic acid was obtained as a brown solid (213 MBq; 0.383 mmol), 77% radiochemically pure. The radiochemical purity was assessed by radio-HPLC (on C-18 reverse phase column along with eluants as mixtures of water-acetonitrile-trifluoroacetic acid from 90:10:0.1 to 10:90:0.1 by volume, linear gradient over 15 minutes and 5 minutes of isocratic elution, detection wavelength=255 nm, radiometric detection=homogeneous with a 500 μl cell and scintillation cocktail to HPLC effluent ratio of 2:1 by volume), the retention time of title compound (Rt=7.36 minutes) was the same as the retention time of an authentic non-labelled sample. The radiochemical yield of this step was about 29%.

### EXAMPLE 4

### Preparation of (Z)-3-[2,4-dimethyl-5-(2-oxo-1,2-dihydro-indol-3-ylidene[¹⁴C]methyl)-1H-pyrrol-3-yl]-propionic acid ([¹⁴C]SU 6668)

3-(3,5-dimethyl-2-[¹⁴C]formyl-1H-pyrrol-4-yl)]-propionic acid (213 MBq; 0.295 mmol, for instance prepared as described in example 3) and oxindole (46 mg; 0.35 mmol) were dissolved with ethanol (2 ml) then pyrrolidine (40 μl; 0.977 mmol) was added and the solution was stirred at reflux for 90 minutes in the dark. At the end of reaction checked by radio-HPLC (on C-18 reverse phase column along with eluants as mixtures of water-acetonitrile-trifluoroacetic acid from 90:10:0.1 to 10:90:0.1 by volume, linear gradient over 15 minutes and 5 minutes of isocratic elution, detection wavelength=255 nm, radiometric detection=homogeneous with a 500 μl cell and scintillation cocktail to HPLC effluent ratio of 2:1 by volume) the mixture was cooled to room temperature, evaporated under vacuum, diluted with water (300 ml) and added with a 1 N solution of hydrochloric acid up to pH 2. The solution was transferred into a separating funnel and extracted with ethyl acetate (3×100 ml). The collected organic phases were pooled, washed with brine (2×100 ml) and after evaporation to dryness under vacuum, the crude (Z)-3-[2,4-dimethyl-5-(2-oxo-1,2-dihydro-3H-indol-3-ylidene[¹⁴C]methyl)-1H-pyrrol-3-yl]-propionic acid ([¹⁴C]SU 6668) was obtained (171.5 MBq; 0.309 mmol) 84% radiochemically pure. The purity was assessed by radio-HPLC (on C-18 reverse phase column along with eluants as mixtures of water-acetonitrile-trifluoroacetic acid from 90:10:0.1 to 10:90:0.1 by volume, linear gradient over 15 minutes and 5 minutes of isocratic elution, detection wavelength=255 nm, radiometric detection=homogeneous with a 500 μl cell and scintillation cocktail to HPLC effluent ratio of 2:1 by volume), the retention time of title compound (Rt=12.5 minutes) was the same as the retention time of an authentic non-labelled sample. The crude [¹⁴C]SU 6668 with a radiochemical purity of about 84% (prepared as above described) was dissolved in a mixture DMSO: mobile phase A (1:2 by volume) up to a concentration of about 6.5 mg/ml and the solution was protected from light.

Aliquots of about 5 ml of the above solution were injected into the preparative HPLC system (on C-18 reverse phase column along with eluants as mixtures of water-acetonitrile-trifluoroacetic acid (A)90:10:0.1 and (B)10:90:0.1 by volume, isocratic for 25 minutes at 75% A-25% B, linear gradient over 5 minutes up to 100% B and 10 minutes of isocratic elution at 100% B, detection wavelength=254 nm). The real time UV-profile plot of the run was followed by sight to identify the [¹⁴C]SU 6668 peak. The column eluate corresponding to the pure [¹⁴C]SU 6668 was collected in a glass flask protected from light. The fractions containing the compound were combined and acetonitrile was removed by evaporation. The acidic aqueous solution was transferred into a separating funnel and extracted with ethyl acetate (1×200 ml). The organic phase was separated, washed with brine (1×200 ml) and after solvent evaporation, [¹⁴C]SU 6668 was obtained (98.23 MBq; 0.177 mmol) 99% radiochemically pure. The radiochemical purity was assessed by radio-HPLC (on C-18 reverse phase column along with eluants as mixtures of water-acetonitrile-trifluoroacetic acid from 90:10:0.1 to 10:90:0.1 by volume, linear gradient over 15 minutes and S minutes of isocratic elution, detection wavelength=255 nm, radiometric detection—homogeneous with a 500 μl cell and scintillation cocktail to RPLC effluent ratio of 2:1 by volume), the retention time of title compound (Rt=12.5 minutes) was the same as the retention time of an authentic non-labelled sample. The mass spectrum of the title compound was recorded using the electrospray ionization technique (ESI) with positive ion detection. The ESI mass spectrum showed the protonated molecular ions at m/z 311 amu of (Z)-3-[2,4-dimethyl-5-(2-oxo-1,2-dihydro-3H-indol-3-ylidene[¹⁴C]methyl)-1H-pyrrol-3-yl]-propionic acid and also at m/z 309 amu of (Z)-3-[2,4-dimethyl-5-(2-oxo-1,2-dihydro-3H-indol-3-ylidenemethyl)-1H-pyrrol-3-yl]-propionic acid. The radiochemical yield of this step including the purification was about 46%.

### EXAMPLE 5

### Preparation of 5-[¹⁴C]formyl-2,4-dimethyl-1H-pyrrole-3-carboxylic acid

Dimethyl[¹⁴C]formamide (about 740 MBq, 1.309 mmol) was cooled-with an ice bath and very slowly added via syringe with diphosphoryl chloride (DPC 97%; 500 μl). After 10 minutes of stirring, the above cooled (ice bath) solution was added with ethyl 2,4-dimethyl-1H-pyrrole-3-carboxylate (278 mg, 1.66 mmol) over 15 minutes under nitrogen and then allowed to warm to room temperature. After 30 minutes a check of the reaction mixture (by radio-HPLC on C-18 reverse phase column along with eluants as mixtures of water-acetonitrile-trifluoroacetic acid from 90:10:0.1 to 10:90:0.1 by volume, linear gradient over 15 minutes and 5 minutes of isocratic elution, detection wavelength=255 nm, radiometric detection=homogeneous with a 500 μl cell and scintillation cocktail to HPLC effluent ratio of 2:1 by volume) showed the complete disappearance of [¹⁴C]-DMF. The brown solution was cooled again (ice bath), diluted with a mixture of water:methanol (5:1 by volume; 1 ml), transferred into a cooled (ice bath) round-bottomed flask, added with further water:methanol (5:1 by volume; 4 ml) and adjusted to pH 7 by adding a 10% solution of potassium hydroxide. After introduction of an additional amount of the above potassium hydroxide solution (800 μl) into the reaction flask, the ice bath was removed and the white-yellowish suspension was heated at reflux for 4 hours.

After cooling to room temperature, a clear yellow solution with traces of a brown oil on the surface was obtained. The mixture was adjusted to pH <4 by adding a 10% solution of hydrochloric acid under vigorous stirring, thus obtaining an orange-brown suspension which was filtered through a sintered-glass filtering funnel. The brown solid residue was washed in suspension with a 5% solution of hydrochloric acid (2×6 ml) and water until neutral colourless washings were collected (9×7 ml). The yellow solid residue was dissolved in a mixture of ethanol:methanol:dimethylformamide for total activity determination and analytical checks. After solvent evaporation to dryness under vacuum, 5-[¹⁴C]formyl-2,4-dimethyl-1H-pyrrole-3-carboxylic acid (492 MBq) was obtained >92% radiochemically pure and used in the next step without further purification. The radiochemical puritywas assessed by radio-HPLC (on C-18 reverse phase column along with eluants as mixtures of water-acetonitrile-trifluoroacetic acid from 90:10:0.1 to 10:90:0.1 by volume, linear gradient over 15 minutes and 5 minutes of isocratic elution, detection wavelength=255 nm, radiometric detection=homogeneous with a 500 μl cell and scintillation cocktail to HPLC effluent ratio of 2:1 by volume), the retention time of title compound (Rt=6.6 minutes) was the same as the retention time of an authentic non-labelled sample. The radiochemical yield of the step was about 66%.

### EXAMPLE 6

### Preparation of N-[2-(diethylamino)ethyl]-5-[¹⁴C]formyl-2,4-dimethyl-1H-pyrrole-3-carboxamide

Benzotriazol-1-ylotris(dimethylamino)phosphonium hexafluorophosphate (BOP, 1 g, 2.26 mmol), triethylamine (480 μl, 3.43 mmol) and N,N-diethylethane-1,2-diamine (360 μl, 2.56 mmol) were slowly added under nitrogen with stirring to a cooled (ice bath) solution of 5-[¹⁴C]formyl-2,4-dimethyl-1H-pyrrole-3-carboxylic acid (167 mg, 455 MBq, 0.1 mmol, for instance prepared as described in example 5, in DMF (5 ml). The ice bath was removed and the reaction mixture was stirred at room temperature for 40 minutes. At the end of the reaction (checked by radio-HPLC on C-18 reverse phase column along with eluants as mixtures of water-acetonitrile-trifluoroacetic acid from 90:10:0.1 to 10:90:0.1 by volume, linear gradient over 15 minutes and 5 minutes of isocratic elution, detection wavelength=255 nm, radiometric detection=homogeneous with a 500 μl cell and scintillation cocktail to HPLC effluent ratio of 2:1 by volume) the mixture was diluted with water (200 ml) and added with a 10% solution of hydrochloric acid (40 ml). After 10 minutes stirring, the acidic solution was transferred into a separating funnel and washed with ethyl acetate (3×100 ml). The aqueous phase was adjusted to pH >12 by adding a 10% solution of potassium hydroxide and extracted with ethyl acetate (3×80 ml). The collected organic phases were pooled, washed with brine (3×70 ml), dried over sodium sulfate and, after filtration, evaporated to dryness under vacuum. After solvent evaporation to dryness under vacuum, N-[2-(diethylamino)ethyl]-5-[¹⁴C]formyl-2,4-dimethyl-1H-pyrrole-3-carboxamide (326 MBq) was obtained >95% radiochemically pure and used in the next step without further purification. The radiochemical purity was assessed by radio-HPLC (on C-18 reverse phase column along with eluants as mixtures of water-acetonitrile-trifluoroacetic acid from 90:10:0.1 to 10:90:0.1 by volume, linear gradient over 15 minutes and 5 minutes of isocratic elution, detection wavelength=255 nm, radiometric detection=homogeneous with a 500 μl cell and scintillation cocktail to HPLC effluent ratio of 2:1 by volume), the retention time of title compound (Rt=4.9 minutes) was the same as the retention time of an authentic non-labelled sample. The radiochemical yield of this step was about 72%.

### EXAMPLE 7

### Preparation of N-[2-(diethylamino) ethyl]-5-[(Z)-(5-fluoro-2-oxo-1,2-dihydro-3H-indol-3-ylidene)-[¹⁴C]methyl]-2,4-dimethyl-1H-pyrrole-3-carboxamide ([¹⁴C]SU 11248)

5-Fluoro-1,3-dihydro-2H-indol-2-one (137 mg, 0.91 mmol) was added at room temperature under nitrogen with stirring to a suspension of N-[2-(diethylamino)ethyl]-5-[¹⁴C]formyl-2,4-dimethyl-1H-pyrrole-3-carboxamide (190 mg, 326 MBq, 0.71 mmol, for instance prepared as described in example 6, in ethanol (3 ml). A brown clear solution was obtained and, after addition of pyrrolidine (100 μl, 1.2 mmol), the reaction mixture was refluxed for 30 minutes. At the end of reaction (checked by radio-HPLC on C-18 reverse phase column along with eluants as mixtures of water-acetonitrile-trifluoroacetic acid from 90:10:0.1 to 10:90:0.1 by volume, linear gradient over 15 minutes and 5 minutes of isocratic elution, detection wavelength=255 nm, radiometric detection=homogeneous with a 500 μl cell and scintillation cocktail to HPLC effluent ratio of 2:1 by volume) the mixture was cooled to room temperature, evaporated under vacuum, diluted with water (300 ml) and added with a 10% solution of hydrochloric acid (50 ml). The obtained clear brown solution was washed with ethyl acetate (5×80 ml), adjusted to pH >12 by adding a 10% solution of potassium hydroxide and extracted with ethyl acetate (7×50 ml). The collected organic phases were pooled, washed with brine (3×70 ml) and concentrated under vacuum for activity determination and analytical checks. The solution was evaporated to dryness under vacuum obtaining N-[2-(diethylamino) ethyl]-5-[(Z)-(5-fluoro-2-oxo-1,2-dihydro-3H-indol-3-ylidene)-[¹⁴C]methyl]-2,4-dimethyl-1H-pyrrole-3-carboxamide ([¹⁴C]SU 11248) (240 M3q) as a yellow-orange solid >97% radiochemically pure. The purity was assessed by radio-HPLC (on C-18 reverse phase column along with eluants as mixtures of water-acetonitrile-trifluoroacetic acid from 90:10:0.1 to 10:90:0.1 by volume, linear gradient over 15 minutes and 5 minutes of isocratic elution, detection wavelength=255 nm, radiometric detection=homogeneous with a 500 μl cell and scintillation cocktail to HPLC effluent ratio of 2:1 by volume), the retention time of title compound (Rt=9.8 minutes) was the same as the retention time of an authentic non-labelled sample. The mass spectrum of the title compound was recorded using the electrospray ionization technique (ESI) with positive ion detection. The ESI mass spectrum showed the protonated molecular ions at m/z 411 amu of N-[2-(diethylamino)ethyl]-S-[(Z)-(5-fluoro-2-oxo-1,2-dihydro-3H-indol-3-ylidene)-[¹⁴C]methyl]-2,4-dimethyl-1H-pyrrole-3-carboxamide and also at m/z 409 amu of N-[2-(diethylamino)ethyl]-5-[(Z)-(5-fluoro-2-oxo-1,2-dihydro-3H-indol-3-ylidene)-methyl]-2,4-dimethyl-1H-pyrrole-3-carboxamide. The radiochemical yield of this step was about 74%.

### EXAMPLE 8

### Preparation of 3-(2-[¹⁴C]formyl-5-methyl-1H-pyrrol-3-yl)propanoic acid

Dimethyl[¹⁴C]formamide (about 740 MBq, 1.045 mmol) was cooled with an ice bath and very slowly added via a syringe with diphosphoryl chloride (DPC 97%, 800 μl). After 10 minutes of stirring, the above cooled solution was added with 3-(5-methyl-1H-pyrrol-3-yl)propanoic acid (66.7 mg; 0.435 mmol) over 15 minutes under nitrogen, then allowed to warm to room temperature and the mixture was stirred for 30 minutes at room temperature.

At the end of the reaction, checked by radio-HPLC (as formerly indicated in previous examples), the mixture was cooled to 0° C. and a solution at this same temperature of methanol:water=1:5 (v:v, 1 ml) was slowly added therein.

After adjusting the pH to 12 by addition of potassium hydroxide (45%), the solution was stirred at room temperature for 30 minutes. The obtained suspension was filtered through a D4 sintered glass filter thus obtaining a clear yellow solution which was cooled to 0° C. and then added with a 10 N solution of hydrochloric acid up to pH 3. The mixture was stirred at 0° C. for 15 minutes and the resulting suspension was filtered through a D4 sintered glass filter. The filtered brown solution was transferred into a separating funnel and extracted with ethyl acetate (4×25 ml). The combined organic phases were washed with brine (1×100 ml) then dried over IST phase and, after evaporation to dryness under vacuum, the title compound was obtained as a brown solid (279 MBq; 0.134 mmol). The radiochemical yield of this step was about 38%.

### EXAMPLE 9

### Preparation of 3-{5-methyl-2-[(Z)-(2-oxo-1,2-dihydro-3H-indol-3-ylidene) [¹⁴C]methyl]-1H-pyrrol-3-yl}propanoic acid ([¹⁴C]SU-10944)

3-(2-[¹⁴C]formyl-5-methyl-1H-pyrrol-3-yl)propanoic acid (279 MBq; 0134 mmol) and oxindole (19.6 mg; 0.147 mmol) were dissolved with ethanol (2 ml); pyrrolidine (30 μl; 0.35 mmol) was then added therein and the solution was stirred at reflux for 90 minutes in the dark. At the end of the reaction, checked by radio-HPLC (as formerly indicated in previous examples), acetic acid (30 μl) was added and the solution was stirred at reflux for 5 minutes in the dark. The reaction mixture was cooled to room temperature, evaporated to dryness under vacuum and dissolved with a 1 N solution of potassium hydroxide (5 ml). The solution was then transferred into a separating funnel, washed with ethyl acetate (3×8 ml), added with a 10 N solution of hydrochloric acid up to pH 3 and then extracted with ethyl acetate (4×10 ml). The combined organic phases were washed with brine (1×50 ml), dried over IST phase column and, after evaporation to dryness under vacuum, the title crude compound was obtained (240.87 MBq; 0.116 mmol; 94% radiochemically pure).

### EXAMPLE 10

Purification of ([¹⁴C]SU-10944)

The crude ([¹⁴C]SU-10944) with a radiochemical purity of about 94%, as obtained according to example 9, was dissolved in dimethylsulfoxide up to a concentration of about 11 mg/ml, in the dark.

Aliquots of about 3 ml of the above solution were injected into a preparative HPLC system (see below):

| column: | Xterra MS C18; 100 × 30 mm ID (5 μM); | | | |
|---|---|---|---|---|
| column | room temperature; | | | |
| temperature: | | | | |
| injection volume: | 3 ml; | | | |
| sample diluent: | DMSO; | | | |
| mobile phase A: | acetonitrile:water:trifluoroacetic acid = | | | |
| | 10:90:0.1 by volume; | | | |
| mobile phase B: | acetonitrile:water:trifluoroacetic acid = | | | |
| | 90:10:0.1 by volume; | | | |
| elution: | time interval (min); | pump condition; | % A; | % B |
| | 0 | ready-to-run | 100 | 0 |
| | 15 | linear gradient | 0 | 100 |
| | 3 | isocratic | 0 | 100 |
| | 1 | reequilibration, | 100 | 0 |
| | | gradient | | |

| | |
|---|---|
| mobile phase | 45 ml /min |
| flow rate: | |
| UV detection: | 254 nm; sampling rate at least 2 p · ts/sec. |

The real time UV-profile plot of the run was followed visually so as to identify the [¹⁴C]SU 10944 peak.

The column eluate corresponding to the pure compound was collected in a glass flask protected from light. The fractions containing the compound were combined and acetonitrile was removed by evaporation. The acidic aqueous solution was transferred into a separating funnel and extracted with ethyl acetate (1×100 ml). The collected organic phase was washed with brine (1×50 ml), dried over IST phase separating columns and, after solvent evaporation to dryness, the title compound was obtained with a radiochemical purity >97% (159 MBq; 0.076 mmol).

### EXAMPLE 11

### Preparation of 5-[¹⁴C]formyl-2,4-methyl-1H-pyrrole-3-carboxylic acid[(2S)-2-hydroxy-3-morpholin-4-yl-propyl]amide

Benzotriazol-1-yloxytris(dimethylamino)phosphonium hexa-fluorophosphate (BOP 97%; 38.2 mg; 0.086 mmol), triethylamine (15 μl; 0.108 mmol) and (2S)-1-amino-3-morpholin-4-yl-propan-2-ol (13.60 mg; 0.085 mmol) were slowly added, under nitrogen with stirring, to a cooled (ice bath) solution of 5-[¹⁴C]formyl-2,4-dimethyl-1H-pyrrole-3-carboxylic acid (5.3 mg; 72.89 MBq; 0.031 mmol) in dimethylformamide (2 ml), being prepared as described in example 5.

The ice bath was removed and the reaction mixture was stirred at room temperature for 40 minutes. At the end of the reaction, checked by radio-HPLC as reported in previous examples, the mixture was diluted with water (20 ml) and acidified with a 6 N solution of hydrochloric acid up to pH 3. After 10 minutes stirring, the acidic solution was transferred into a separating funnel and washed with ethyl acetate (3×20 ml). The aqueous phase was adjusted to pH >12 by adding a 45% solution of potassium hydroxide and extracted with ethyl acetate (4×20 ml). The collected organic phases were pooled, washed with brine (1×50 ml), dried over sodium sulfate and, after filtration, evaporated to dryness under vacuum. The orange oily residue was dissolved in ethyl acetate (20 ml) for total activity determination and analytical checks. The solution was evaporated to dryness under vacuum thus affording the title compound (39.83 MBq; >72% radiochemically pure. The obtained compound (radiochemical yield of about 54%) was used in the subsequent step without further purification.

### EXAMPLE 12

### Preparation of 5-[(Z)-(5-fluoro-2-oxo-1,2-dihydro-3H-indol-3-ylidene)[¹⁴C]methyl]-N-[(2S)-2-hydroxy-3-morpholin-4-ylpropyl]-2,4-dimethyl-1H-pyrrole-3-carboxamide ([¹⁴C]SU-14813)

5-[¹⁴C]formyl-2,4-dimethyl-1H-pyrrole-3-carboxylic acid[(2S)-2-hydroxy-3-morpholin-4-yl-propyl]amide (23.45 MBq; 0.011 mmol) and 5-fluoro-1,3-dihydro-indol-2-one (2.2 mg; 0.0145 mmol) were dissolved in ethanol (1.5 ml). Pyrrolidine (5 μl; 0.06 mmol) was then added therein and the solution was stirred at reflux for 40 minutes in the dark. At the end of reaction, checked by radio-HPLC as reported in previous examples, the mixture was cooled to room temperature, evaporated to dryness under vacuum and dissolved with a 1 N solution of potassium hydroxide (20 ml). The solution was then transferred into a separating funnel and extracted with ethyl acetate (3×20 ml). The combined organic phases were washed with brine (1×50 ml), dried over IST phase separating columns and, after evaporation to dryness under vacuum, the crude title compound was obtained (12.60 MBq; 0.006 mmol; 82% radiochemically pure). The radiochemical yield of this step was of about 54%.

### EXAMPLE 13

Purification of ([¹⁴C]SU-14813)

The crude ([¹⁴C]SU-14813) with a radiochemical purity of about 82%, being prepared according to example 12, was dissolved in methanol:mobile phase A=1:2 (v/v) up to a concentration of about 1.9 mg/ml, in the dark.

Aliquots of about 3 ml of the above solution were injected into a preparative HPLC system (see below):

| column: | Xterra MS C18; 100 × 30 mm ID (5 μM); | | | |
|---|---|---|---|---|
| column | room temperature; | | | |
| temperature: | | | | |
| injection volume: | 3 ml; | | | |
| sample diluent: | methanol:mobile phase A = 1:2 (v/v); | | | |
| mobile phase A: | acetonitrile:water:trifluoroacetic acid = | | | |
| | 10:90:0.1 by volume; | | | |
| mobile phase B: | acetonitrile:water:trifluoroacetic acid = | | | |
| | 90:10:0.1 by volume; | | | |
| elution: | time interval (min); | pump condition; | % A; | % B |
| | 0 | ready-to-run | 100 | 0 |
| | 15 | linear gradient | 0 | 100 |
| | 3 | isocratic | 0 | 100 |
| | 1 | reequilibration, | 100 | 0 |
| | | gradient | | |

| | |
|---|---|
| mobile phase | 45 ml/min |
| flow rate: | |
| UV detection: | 254 nm; sampling rate at least 2 p · ts/sec. |

The real time UV-profile plot of the run was followed visually so as to identify the [¹⁴C]SU-14813 peak.

The column eluate corresponding to the pure compound was collected in a glass flask protected from light. The fractions containing the compound were combined and acetonitrile was removed by evaporation. The obtained aqueous solution was transferred into a separating funnel and adjusted to pH=12 by adding a 45% solution of potassium hydroxide and extracted with ethyl acetate (1×50 ml).

The collected organic phase was washed with brine (1×50 ml), dried over IST phase separating columns and, after solvent evaporation to dryness, the title compound was obtained with a radiochemical purity >97% (11.16 MBq; 0.005 mmol).

1. A compound of general formula (I) below: 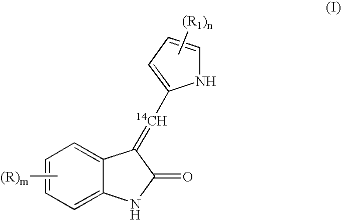
wherein
each R group is, at one or more of the positions 4, 5, 6 and 7 of the indolinone ring and independently from each other, a straight or branched C₁-C₄ alkyl or alkoxy group or a halogen atom;
each R₁ group is, the same or different and at one or more of the positions of the pyrrole ring, a C₁-C₄ alkyl or a group of general formula —(CH₂)ₚCO₂R′, —(CH₂)ₚ—CONR′R″ or —CONH—(CH₂)ₚ—CONR′R″ wherein p is 0, 1, 2 or 3, the alkylene —(CH₂)ₚ— chain is optionally substituted by hydroxy, and R′ and R″ are selected, each independently, from hydrogen or straight or branched C₁-C₄ alkyl optionally substituted by hydroxy or, taken together with the nitrogen atom to which they are attached, R′ and R″ may form a pyrrolidino, piperidino or morpholino group;
m is 0 or an integer from 1 to 4;
n is 0 or an integer from 1 to 3;
or pharmaceutically acceptable salts thereof.

2. A compound according to claim 1 wherein the pyrrole ring is substituted by one or more of the groups selected from methyl, carboxy, ethoxycarbonyl, carboxyethyl, N,N-diethyl-aminocarbonyl, N-[(2-diethylamino)ethyl]carboxamide or N-[2-hydroxy-3-morpholin-4-ylpropyl]carboxamide.

3. A compound according to claim 1 which is 3-((3,5-dimethyl-1H-pyrrol-2-yl)[¹⁴C]methylene-1,3-dihydro-2H-indol-2-one; 5-[(1,2-dihydro-2-oxo-3H-indol-3ylidene) [¹⁴C]methyl]-2,4-dimethyl-1H-pyrrole-3-propionic acid; N-[(2-diethylamino)ethyl]-5-[(5-fluoro-1,2-dihydro-2-oxo-3H-indol-3-ylidene)[¹⁴C]methyl]-2,4-dimethyl-1H-pyrrole-3-carboxamide; 3-{5-methyl-2-[(Z)-(2-oxo-1,2-dihydro-3H-indol-3-ylidene)[¹⁴C]methyl]-1H-pyrrol-3-yl)}propanoic acid; and 5-[(Z)-(5-fluoro-2-oxo-1,2-dihydro-3H-indol-3-ylidene) [¹⁴C]methyl]-N-[(2S)-2-hydroxy-3-morpholin-4-ylpropyl]-2,4-dimethyl-1H-pyrrole-3-carboxamide.

4. A process for preparing a compound of formula (I) according to claim 1 which process comprises:
a) reacting dimethyl-[¹⁴C]formamide with-a suitable pyrrole derivative of formula (II), in the presence of diphosphoryl-chloride 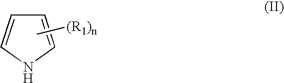
wherein R₁ and n are as defined in claim 1, so as to obtain a compound of formula (III) 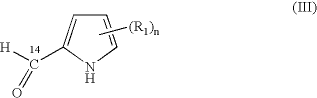
and optionally converting a compound of formula (III) into another compound of formula (III);
b) reacting under basic conditions the compound of formula (III) with an oxindole derivative of formula (IV) 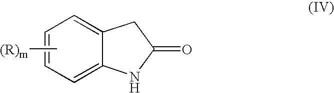
wherein R and m are as defined in claim 1, so as to obtain a compound of formula (I) and, optionally converting it into another compound of formula (I) and/or into a pharmaceutically acceptable salt thereof.

5. A process according to claim 4 wherein, in step (b), basic conditions are obtained by means of pyrrolidine.

6. A compound of formula (IIIa) or (IIIb) below 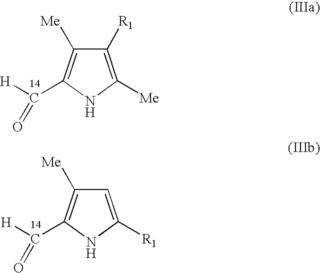
wherein R₁ is a hydrogen atom or a group selected from —(CH₂)₂—CO₂H, —CO₂H, —CO₂CH₂CH₃, —CONH—(CH₂)₂—N(CH₂CH₃)₂ and

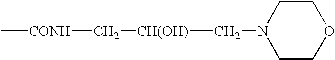

7. Use of a compound of formula (I), as defined in claim 1, for absorption, distribution, metabolism and excretion (ADME) studies.
